(19) United States
(12) Patent Application Publication
(10) Pub. No.: US 2016/0144072 A1
(43) Pub. Date: May 26, 2016

(54) Mesh Fiber Members and Methods for Forming and Using Same for Treating Damaged Biological Tissue

(71) Applicant: CorMatrix Cardiovascular, Inc., Roswell, GA (US)

(72) Inventors: Robert G. Matheny, Norcross, GA (US); Derek S. Francis, Oakland, CA (US)

(21) Appl. No.: 14/554,847

(22) Filed: Nov. 26, 2014

(51) Int. Cl. A61L 27/36; A61L 27/54; A61L 27/38; A61L 27/58

(52) U.S. Cl. CPC A61L 27/3633; A61L 27/58; A61L 27/3629; A61L 27/54; A61L 27/3804; A61L 27/3834; A61L 2300/21; A61L 2430/20; A61L 2300/414; A61L 2300/252; A61L 2300/404; A61L 2300/402; A61L 2300/41; A61L 2300/416; A61L 2300/40; A61L 2300/434; A61L 2300/254; A61L 2300/42; A61L 2430/00

(57) ABSTRACT

A mesh fiber member having a plurality of biodegradable fibers, the mesh fiber member being configured to induce modulated healing of damaged biological tissue when deployed proximate thereto. The strands comprise an extracellular matrix (ECM) composition or an ECM-mimicking biomaterial composition, such as poly(glycerol sebacate) (PGS), and can include a biodegradable ECM, polymeric or ECM-mimicking biomaterial composition coating.

### FIELD OF THE INVENTION

The present invention relates to implantable biological prostheses for treating biological tissue. More particularly, the present invention relates to non-antigenic, resilient, biocompatible biological prostheses, i.e. mesh constructs, that can be engineered into a variety of shapes and used to treat, augment, or replace damaged or diseased biological tissue.

### BACKGROUND OF THE INVENTION

As is well known in the art, tissue prostheses or grafts are often employed to treat or replace damaged or diseased biological tissue. However, despite the growing sophistication of medical technology, the use of grafts to treat or replace damaged biological tissue remains a frequent and serious problem in health care. The problem is often associated with the materials employed to construct the grafts.

As is also well known in the art, the optimal graft material should be chemically inert, non-carcinogenic, capable of resisting mechanical stress, capable of being fabricated in the form required, and sterilizable. Further, the material should be resistant to physical modification by tissue fluids, and not excite an inflammatory reaction, induce a state of allergy or hypersensitivity, or, in some cases, promote visceral adhesions. See, e.g., Jenkins, et al., Surgery, vol. 94(2), pp. 392-398 (1983).

Various materials and/or structures have thus been employed to construct grafts that satisfy the aforementioned optimal characteristics, including tantalum gauze, stainless mesh, Dacron®, Orlon®, Fortisan®, nylon, knitted polypropylene (e.g., Marlex®), microporous expanded-polytetrafluoroethylene (e.g., Gore-Tex®), Dacron reinforced silicone rubber (e.g., Silastic®), polyglactin 910 (e.g., Vicryl®), polyester (e.g., Mersilene®), polyglycolic acid (e.g., Dexon®), processed sheep dermal collagen, crosslinked bovine pericardium (e.g., Peri-Guard®), and preserved human dura (e.g., Lyodura®).

As discussed in detail below, although some of the noted graft materials satisfy some of the aforementioned optimal characteristics, few, if any, satisfy all of the optimal characteristics.

The major advantages of metallic meshes, e.g., stainless steel meshes, are that they are inert, resistant to infection and can stimulate fibroplasia. Several major disadvantages are fragmentation, which can, and in many instances will, occur after the first year of administration, and the lack of malleability.

Synthetic meshes have the advantage of being easily molded and, except for nylon, retain their tensile strength in or on the body. In European Patent No. 91122196.8 a triple-layer vascular prosthesis is disclosed that utilizes non-resorbable synthetic mesh as the center layer. The synthetic textile mesh layer is used as a central frame to which layers of collagenous fibers are added, resulting in the tri-layered prosthetic device.

There are several drawbacks and disadvantages associated with non-resorbable synthetic mesh. Among the major disadvantages are the lack of inertness, susceptibility to infection, and interference with wound healing.

In contrast to non-resorbable synthetic meshes, absorbable synthetic meshes have the advantage of impermanence at the deployment site, but often have the disadvantage of loss of mechanical strength (as a result of dissolution by the host) prior to adequate cell and tissue ingrowth.

The most widely used graft material for abdominal wall replacement and for reinforcement during hernia repairs is Marlex®, i.e. polypropylene. A major disadvantage associated with polypropylene mesh grafts is that with scar contracture, polypropylene mesh grafts become distorted and separate from surrounding normal tissue.

Gore-Tex®, i.e. polytetrafluoroethylene, is currently believed to be the most chemically inert graft material. However, a major problem associated with the use of polytetrafluoroethylene is that in a contaminated wound it does not allow for any macromolecular drainage, which limits treatment of infections.

Collagen is another commonly employed graft material. Collagen first gained utility as a material for medical use because it was a natural biological graft substitute that was in abundant supply from various animal sources.

The design objectives for the original collagen grafts were the same as for synthetic polymer grafts, i.e. the grafts should persist and essentially act as an inert material. With these objectives in mind, purification and crosslinking methods were developed to enhance mechanical strength and decrease the degradation rate of the collagen.

The crosslinking agents that were originally used included glutaraldehyde, formaldehyde, polyepoxides, diisocyanates and acyl azides. Glutaraldehyde was also used as a sterilizing agent.

A major disadvantage of crosslinking collagen is, however, that it reduces the antigenicity of the material by linking the antigenic epitopes, rendering them either inaccessible to phagocytosis or unrecognizable by the immune system.

Crosslinking collagen will thus, in general, generate collagenous material that resembled a synthetic material more than a natural biological tissue, both mechanically and biologically.

Tissue prostheses or graft material derived from mammalian tissue, i.e. extracellular matrix (ECM), is also often employed to construct tissue prostheses or grafts. Illustrative are the grafts disclosed in U.S. Pat. No. 3,562,820 (tubular, sheet and strip grafts formed from submucosa adhered together by use of a binder paste, such as a collagen fiber paste, or by use of an acid or alkaline medium), and U.S. Pat. No. 4,902,508 (a three layer tissue graft composition derived from small intestine comprising tunica submucosa, the muscularis mucosa, and stratum compactum of the tunica mucosa).

Although a number of the ECM based tissue prostheses or grafts satisfy many of the aforementioned optimal characteristics, efforts continue to develop improved prostheses and/or grafts that can successfully be employed to replace or to facilitate the repair of biological tissue, such as abdominal wall defects and vasculature, whereby the host's own cells can be optimally exploited in the repair process.

Recent studies have, additionally, suggested that cells rely on spatial cues provided by the ECM collagen structure. While ECM delivery platforms have been found highly effective, native ECM can, and often will, provide random micro- and/or nano-scale structural cues to modulate the aforementioned optimal characteristics.

Various ECM and polymer based apparatus have also been developed in an attempt to modulate visceral adhesion. Illustrative are the ECM and polymer based apparatus, i.e. grafts and endografts, disclosed in U.S. Pat. Nos. 7,244,444, 7,914,808 and Intl. Pub. No. WO 2013/178229. Polymer based apparatus are also disclosed in Liu, et al. “Production of endothelial cell-enclosing alginate-based hydrogel fibers with cell adhesive surface through simultaneous cross-linking by horseradish peroxidase-catalyzed reaction in a hydrodynamic spinning process,” Journal of Bioscience and Bioengineering, Vol. 114, No. 3, pp. 353-359, (2012).

A major drawback of the noted polymer based apparatus, as well as most known apparatus, is that the apparatus often comprise or include a permanent structure that remains in the body, i.e. non-biodegradable. As is well known in the art, such structures (or devices) can, and in most instances will, cause irritation and undesirable biologic responses in the surrounding tissue.

Such structures (and devices) are also prone to failure, resulting in severe adverse consequences, e.g., ruptured vessels.

U.S. Pat. No. 7,244,444 discloses vascular or endoluminal grafts comprising a woven or knitted polymer scaffold having ECM disposed over the surface(s) of the scaffold.

U.S. Pat. No. 7,914,808 similarly discloses a graft scaffold constructed from a mat of fresh or nonwoven synthetic material coated with Small Intestine Submucosa (SIS), a mixture of SIS and a synthetic polymer, or layers of SIS and synthetic polymer.

Intl. Pub. No. WO 2013/178229 discloses a biocompatible nonwoven mesh structure comprising polymeric fibers and interconnected by the glue points.

A major drawback of the noted structures it that they include the use of polymeric materials, which often comprise or include a permanent structure that remains in the body, i.e. non-biodegradable.

There is, thus, a need for biological prostheses, i.e. mesh structures or constructs, that effectively modulates alignment of proliferating cells during tissue remodeling to mimic the native alignment of existing tissue cells.

It is therefore an object of the present invention to provide biological mesh prostheses (referred to herein as “mesh fiber members”) that induce modulated healing, including modulated inflammation of damaged tissue and/or neovascularization, host tissue proliferation, bioremodeling, and regeneration of tissue and associated structures with site-specific structural and functional properties.

### SUMMARY OF THE INVENTION

The present invention is directed to biological prostheses in the form of mesh fiber members that are configured to treat damaged biological tissue and methods for forming and using same.

In some embodiments, the mesh fiber members comprise a bioremodelable fiber or strand.

In some embodiments, the mesh fiber members comprise a plurality of bioremodelable strands, i.e. a fiber construct.

In some embodiments, the mesh fiber members comprise a plurality of fiber constructs.

According to the invention, the strands can be oriented in various configurations to form a fiber construct.

In some embodiments, the fiber construct comprises an intertwined strand configuration.

In some embodiments, the fiber construct comprises an intertwined braided strand configuration.

According to the invention, the biodegradable strands and fiber constructs can similarly be oriented in various configurations, i.e. mesh patterns, to form a mesh fiber member.

In some embodiments, the mesh pattern comprises an intertwined strand and/or fiber construct configuration.

In some embodiments, the mesh pattern comprises an intertwined directionally aligned strand and/or fiber construct configuration.

In some embodiments, the mesh pattern comprises an intertwined substantially perpendicular alignment of strands and/or fiber constructs.

In some embodiments, the mesh pattern comprises intertwined strands and/or fiber constructs that are oriented at an angle in the range of 0-90° relative to the plane defined by a linear axis of the mesh fiber member.

In some embodiments, the mesh pattern comprises a random orientation of strands and/or fiber constructs.

In some embodiments of the invention, the mesh fiber members comprise at least one secured border bound to at least one strand and/or fiber construct.

In some embodiments of the invention, the strands and/or fiber constructs and, hence, mesh fiber members formed therefrom comprise ECM derived from a mammalian tissue source selected from the group comprising, without limitation, the small intestine, large intestine, stomach, lung, liver, kidney, pancreas, placenta, heart, bladder, prostate, tissue surrounding growing enamel, tissue surrounding growing bone, and any fetal tissue from any mammalian organ. The ECM can also comprise collagen from mammalian sources.

In some embodiments of the invention, the strands and/or fiber constructs and, hence, mesh fiber members formed therefrom comprise a biocompatible polymeric composition comprising a polymeric material selected from the group comprising, without limitation, polyglycolide (PGA), polylactide (PLA), polyepsilon-caprolactone (PCL), polydioxanone (a polyether-ester), poly lactide-co-glycolide, polyamide esters, polyalkalene esters, polyvinyl esters, polyvinyl alcohol, and polyanhydrides. Natural polymeric materials, include, without limitation, polysaccharides (e.g. starch and cellulose), proteins (e.g., gelatin, casein, silk, wool, etc.), and polyesters (e.g., polyhydroxyalkanoates).

In some embodiments of the invention, the strands and/or fiber constructs and, hence, mesh fiber members formed therefrom comprise an ECM-mimicking polymeric biomaterial.

In some embodiments, the ECM-mimicking biomaterial comprises poly(glycerol sebacate) (PGS).

In some embodiments of the invention, the strands and/or fiber constructs and, hence, mesh fiber members formed therefrom comprise an ECM-PGS composition.

In some embodiments of the invention, the strands and/or fiber constructs and, hence, mesh fiber members formed therefrom comprise at least one additional biologically active agent, i.e. an agent that induces or modulates a physiological or biological process, or cellular activity, e.g., induces proliferation, and/or growth and/or regeneration of tissue.

In some embodiments, the biologically active agent comprises a cell selected from the group comprising, without limitation, embryonic stem cells, mesenchymal stem cells, hematopoietic stem cells, bone marrow stem cells, bone marrow-derived progenitor cells, myosatellite progenitor cells, totipotent stem cells, pluripotent stem cells, multipotent stem cells, oligopotent stem cells and unipotent stem cells.

In some embodiments, the biologically active agent comprises a growth factor selected from the group comprising, without limitation, transforming growth factor alpha (TGF-α), transforming growth factor beta (TGF-β), fibroblast growth factor-2 (FGF-2), basic fibroblast growth factor (bFGF), vascular epithelial growth factor (VEGF), and insulin-like growth factor (IGF).

In some embodiments, the biologically active agent comprises a protein selected from the group comprising, without limitation, collagen (types I-V), proteoglycans, glycosaminoglycans (GAGs), glycoproteins, cytokines, cell-surface associated proteins, and cell adhesion molecules (CAMs).

In some embodiments of the invention, the strands and/or fiber constructs and, hence, mesh fiber members formed therefrom further comprise at least one pharmacological agent or composition, i.e. an agent, drug, compound, composition of matter or mixture thereof, including its formulation, which provides some therapeutic, often beneficial, effect.

In a preferred embodiment, the pharmacological agent or composition is selected from the group comprising, without limitation, antibiotics, anti-arrhythmic agents, anti-viral agents, analgesics, steroidal anti-inflammatories, non-steroidal anti-inflammatories, anti-neoplastics, anti-spasmodics, modulators of cell-extracellular matrix interactions, proteins, hormones, growth factors, matrix metalloproteinases (MMPs), enzymes and enzyme inhibitors, anticoagulants and/or antithrombic agents, DNA, RNA, modified DNA and RNA, NSAIDs, and inhibitors of DNA, RNA or protein synthesis.

In some embodiments of the invention, the strands and/or fiber constructs and, hence, mesh fiber members formed therefrom provide a single-stage agent delivery profile, i.e. comprise a single-stage agent delivery vehicle, wherein a modulated dosage of an aforementioned biologically active and/or pharmacological agent is provided.

In some embodiments of the invention, the strands and/or fiber constructs and, hence, mesh fiber members formed therefrom provide a multi-stage agent delivery profile, i.e. comprise a multi-stage delivery vehicle, wherein a plurality of the aforementioned biologically active and/or pharmacological agents are administered via a modulated dosage.

In some embodiments, the strands and/or fiber constructs and, hence, mesh fiber members formed therefrom further comprise at least one coating that includes at least one of the aforementioned biologically active or pharmacological agents.

According to the invention, the coating can be applied to the individual strands, fiber constructs and/or a region of or the entire mesh fiber member.

In some embodiments, the coating comprises an ECM composition.

In some embodiments, the coating comprises a polymeric composition.

In some embodiments, the coating comprises an ECM-PGS composition.

In another embodiment of the invention, there is provided a method of forming the aforementioned mesh fiber members of the invention.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a perspective sectional view of one embodiment of a strand, in accordance with the invention;

FIG. 2 is a perspective sectional view of another embodiment of the strand shown in FIG. 1, in accordance with the invention;

FIG. 3 is a perspective sectional view of a fiber construct, in accordance with the invention;

FIGS. 4-7 are top plan views of several embodiments of the mesh fiber members, in accordance with the invention;

FIG. 8 is a graphical illustration reflecting the effect of a statin augmented ECM on MCP-1 mRNA expression over time, in accordance with the invention;

FIG. 9 is a graphical illustration reflecting the effect of a statin augmented ECM on CCR2 mRNA expression over time, in accordance with the invention;

FIG. 10 is a graphical illustration reflecting the effect of a statin augmented ECM on RAC1 mRNA expression over time, in accordance with the invention; and

FIG. 11 is a graphical illustration reflecting the effect of a statin augmented ECM on MCP-1 concentration and mRNA expression over time, in accordance with the invention.

### DETAILED DESCRIPTION OF THE PREFERRED EMBODIMENT

Before describing the present invention in detail, it is to be understood that this invention is not limited to particularly exemplified apparatus, systems, materials, compositions, structures or methods as such may, of course, vary. Thus, although a number of apparatus, systems, materials, compositions, structures and methods similar or equivalent to those described herein can be used in the practice of the present invention, the preferred apparatus, systems, materials, compositions, structures and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

As used in this specification and the appended claims, the singular forms “a, “an” and “the” include plural referents unless the content clearly dictates otherwise. Thus, for example, reference to “an active” includes two or more such actives and the like.

Further, ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

It is also understood that there are a number of values disclosed herein, and that each value is also herein disclosed as “approximately” that particular value in addition to the value itself. For example, if the value “10” is disclosed, then “approximately 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed then “less than or equal to 10”, as well as “greater than or equal to 10” is also disclosed.

### DEFINITIONS

The terms “prosthesis” and “mesh fiber member” are used interchangeably herein, and mean and include a structure or system that is configured for placement on biological tissue on or in an organ, such as a lumen or vessel. As discussed in detail herein, upon placement of a biological prostheses or mesh fiber member of the invention to biological tissue; particularly, damaged or diseased tissue, the mesh fiber member induces “modulated healing”, as defined herein.

The term “biocompatible”, as used herein, means a device or material that is substantially non-toxic in an in vivo environment, and is not substantially rejected by a recipient's physiological system, i.e. non-antigenic.

The term “anisotropic”, as used herein, means a member having physical properties or characteristics that are directionally dependent.

The terms “mesh” and “mesh structure” are used interchangeably herein, and mean and include a structure comprising at least one fiber or strand or fiber construct, as defined herein, and, in some embodiments, a plurality of interdependent fibers or strands or fiber constructs of a biocompatible material defining a layer of material having a predetermined range of permeability and/or porosity, and configured to be disposed proximate biological tissue. As discussed in detail herein, the “mesh structure” can comprise various configurations of fibers and/or strands and/or fiber construct to provide various desired mesh patterns.

The term “woven”, as used herein, means and includes an ordered arrangement of fibers and/or fiber constructs bonded by physical, mechanical, or chemical means.

The term “nonwoven”, as used herein, refers to an arrangement of fibers and/or fiber constructs bonded by random and/or semi-random entanglement, and/or physical, mechanical or chemical means as opposed to weave or knitted fabrics where the structure is highly ordered. The orientation of the fibers in a nonwoven can be either random or have some degree of order.

The terms “extracellular matrix” and “ECM” are used interchangeably herein, and mean and include a collagen-rich substance that is found in between cells in mammalian tissue, and any material processed therefrom, e.g. decellularized ECM. According to the invention, the ECM material can be derived from various mammalian tissue sources including, without limitation, the small intestine, large intestine, stomach, lung, liver, kidney, pancreas, placenta, heart, bladder, prostate, tissue surrounding growing enamel, tissue surrounding growing bone, and any fetal tissue from any mammalian organ.

The ECM material can thus comprise, without limitation, small intestine submucosa (SIS), urinary bladder submucosa (UBS), stomach submucosa (SS), central nervous system tissue, dermal extracellular matrix, subcutaneous extracellular matrix, gastrointestinal extracellular matrix, i.e. large and small intestines, tissue surrounding growing bone, placental extracellular matrix, ornamentum extracellular matrix, epithelium of mesodermal origin, i.e. mesothelial tissue, cardiac extracellular matrix, e.g., pericardium and/or myocardium, kidney extracellular matrix, pancreas extracellular matrix, lung extracellular matrix, and combinations thereof. The ECM can also comprise collagen from mammalian sources.

The terms “urinary bladder submucosa (UBS)”, “small intestine submucosa (SIS)” and “stomach submucosa (SS)” also mean and include any UBS and/or SIS and/or SS material that includes the tunica mucosa (which includes the transitional epithelial layer and the tunica propria), submucosal layer, one or more layers of muscularis, and adventitia (a loose connective tissue layer) associated therewith.

The ECM can also be derived from basement membrane of mammalian tissue/organs, including, without limitation, bladder, “urinary basement membrane (UBM)”, liver, i.e. “liver basement membrane (LBM)”, and amnion, chorion, allograft pericardium, allograft acellular dermis, amniotic membrane, Wharton's jelly, and combinations thereof.

Additional sources of mammalian basement membrane include, without limitation, spleen, lymph nodes, salivary glands, prostate, pancreas and other secreting glands.

The ECM can also be derived from other sources, including, without limitation, collagen from plant sources and synthesized extracellular matrices, i.e. cell cultures.

The term “angiogenesis”, as used herein, means a physiologic process involving the growth of new blood vessels from pre-existing blood vessels.

The term “neovascularization”, as used herein, means and includes the formation of functional vascular networks that can be perfused by blood or blood components. Neovascularization includes angiogenesis, budding angiogenesis, intussuceptive angiogenesis, sprouting angiogenesis, therapeutic angiogenesis and vasculogenesis.

The term “ECM-mimicking”, as used herein, means and includes a biocompatible and biodegradable biomaterial that induces neovascularization and bioremodeling of tissue in vivo, i.e. when disposed proximate damaged biological tissue. The term “ECM-mimicking” thus includes, without limitation, ECM-mimicking polymeric biomaterials; specifically, poly(glycerol sebacate) (PGS).

The terms “biologically active agent” and “biologically active composition” are used interchangeably herein, and mean and include agent that induces or modulates a physiological or biological process, or cellular activity, e.g., induces proliferation, and/or growth and/or regeneration of tissue.

The terms “biologically active agent” and “biologically active composition” thus mean and include, without limitation, the following growth factors: platelet derived growth factor (PDGF), epidermal growth factor (EGF), transforming growth factor alpha (TGF-α), transforming growth factor beta (TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platelet derived growth factor (PDGF), tumor necrosis factor alpha (TNF-α), and placental growth factor (PLGF).

The terms “biologically active agent” and “biologically active composition” also mean and include, without limitation, embryonic stem cells, mesenchymal stem cells, hematopoietic stem cells, bone marrow stem cells, bone marrow-derived progenitor cells, myosatellite progenitor cells, totipotent stem cells, pluripotent stem cells, multipotent stem cells, oligopotent stem cells and unipotent stem cells. The group also comprises cardiomyocytes, myoblasts, monocytes, parenchymal cells, epithelial cells, endothelial cells, mesothelial cells, fibroblasts, osteoblasts, chondrocytes, exogenous cells, endogenous cells, macrophages, capillary endothelial cells, autologous cells, xenogenic cells, allogenic cells, and cells derived from any of the three germ layers including the endoderm, mesoderm and ectoderm.

The terms “biologically active agent” and “biologically active composition” also mean and include, without limitation, the following biologically active agents (referred to interchangeably herein as a “protein”, “peptide” and “polypeptide”): collagen (types I-V), proteoglycans, glycosaminoglycans (GAGs), glycoproteins, cytokines, cell-surface associated proteins, cell adhesion molecules (CAM), endothelial ligands, matrikines, cadherins, immunoglobins, fibril collagens, non-fibrillar collagens, basement membrane collagens, multiplexins, small-leucine rich proteoglycans, decorins, biglycans, fibromodulins, keratocans, lumicans, epiphycans, heparin sulfate proteoglycans, perlecans, agrins, testicans, syndecans, glypicans, serglycins, selectins, lecticans, aggrecans, versicans, neurocans, brevicans, cytoplasmic domain-44 (CD-44), macrophage stimulating factors, amyloid precursor proteins, heparins, chondroitin sulfate B (dermatan sulfate), chondroitin sulfate A, heparin sulfates, hyaluronic acids, fibronectins, tenascins, elastins, fibrillins, laminins, nidogen/eriactins, fibulin I, fibulin II, integrins, transmembrane molecules, thrombospondins, ostepontins, and angiotensin converting enzymes (ACE).

The terms “pharmacological agent”, “active agent”, “drug” and “active agent formulation” are used interchangeably herein, and mean and include an agent, drug, compound, composition of matter or mixture thereof, including its formulation, which provides some therapeutic, often beneficial, effect. This includes any physiologically or pharmacologically active substance that produces a localized or systemic effect or effects in animals, including warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The terms “pharmacological agent”, “active agent”, “drug” and “active agent formulation” thus mean and include, without limitation, antibiotics, anti-arrhythmic agents, anti-viral agents, analgesics, steroidal anti-inflammatories, non-steroidal anti-inflammatories, anti-neoplastics, anti-spasmodics, modulators of cell-extracellular matrix interactions, proteins, hormones, growth factors, matrix metalloproteinases (MMPs), enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

The terms “pharmacological agent”, “active agent”, “drug” and “active agent formulation” thus include, without limitation, atropine, tropicamide, dexamethasone, dexamethasone phosphate, betamethasone, betamethasone phosphate, prednisolone, triamcinolone, triamcinolone acetonide, fluocinolone acetonide, anecortave acetate, budesonide, cyclosporine, FK-506, rapamycin, ruboxistaurin, midostaurin, flurbiprofen, suprofen, ketoprofen, diclofenac, ketorolac, nepafenac, lidocaine, neomycin, polymyxin b, bacitracin, gramicidin, gentamicin, oyxtetracycline, ciprofloxacin, ofloxacin, tobramycin, amikacin, vancomycin, cefazolin, ticarcillin, chloramphenicol, miconazole, itraconazole, trifluridine, vidarabine, ganciclovir, acyclovir, cidofovir, ara-amp, foscarnet, idoxuridine, adefovir dipivoxil, methotrexate, carboplatin, phenylephrine, epinephrine, dipivefrin, timolol, 6-hydroxydopamine, betaxolol, pilocarpine, carbachol, physostigmine, demecarium, dorzolamide, brinzolamide, latanoprost, sodium hyaluronate, insulin, verteporfin, pegaptanib, ranibizumab, and other antibodies, antineoplastics, anti-VEGFs, ciliary neurotrophic factor, brain-derived neurotrophic factor, bFGF, Caspase-1 inhibitors, Caspase-3 inhibitors, α-Adrenoceptors agonists, NMDA antagonists, Glial cell line-derived neurotrophic factors (GDNF), pigment epithelium-derived factor (PEDF), and NT-3, NT-4, NGF, IGF-2.

The terms “pharmacological agent”, “active agent”, “drug” and “active agent formulation” further mean and include the following Class I-Class V anti-arrhythmic agents: (Class Ia) quinidine, procainamide and disopyramide; (Class Ib) lidocaine, phenytoin and mexiletine; (Class Ic) flecainide, propafenone and moricizine; (Class II) propranolol, esmolol, timolol, metoprolol and atenolol; (Class III) amiodarone, sotalol, ibutilide and dofetilide; (Class IV) verapamil and diltiazem and (Class V) adenosine and digoxin.

The terms “pharmacological agent”, “active agent”, “drug” and “active agent formulation” further mean and include, without limitation, the following antibiotics: aminoglycosides, cephalosporins, chloramphenicol, clindamycin, erythromycins, fluoroquinolones, macrolides, azolides, metronidazole, penicillins, tetracyclines, trimethoprim-sulfamethoxazole and vancomycin.

The terms “pharmacological agent”, “active agent”, “drug” and “active agent formulation” further include, without limitation, the following steroids: andranes (e.g., testosterone), cholestanes, cholic acids, corticosteroids (e.g., dexamethasone), estraenes (e.g., estradiol) and pregnanes (e.g., progesterone).

The terms “pharmacological agent”, “active agent”, “drug” and “active agent formulation” can further include one or more classes of narcotic analgesics, including, without limitation, morphine, codeine, heroin, hydromorphone, levorphanol, meperidine, methadone, oxycodone, propoxyphene, fentanyl, methadone, naloxone, buprenorphine, butorphanol, nalbuphine and pentazocine.

The terms “pharmacological agent”, “active agent”, “drug” and “active agent formulation” can further include one or more classes of topical or local anesthetics, including, without limitation, esters, such as benzocaine, chloroprocaine, cocaine, cyclomethycaine, dimethocaine/larocaine, piperocaine, propoxycaine, procaine/novacaine, proparacaine, and tetracaine/amethocaine. Local anesthetics can also include, without limitation, amides, such as articaine, bupivacaine, cinchocaine/dibucaine, etidocaine, levobupivacaine, lidocaine/lignocaine, mepivacaine, prilocaine, ropivacaine, and trimecaine. Local anesthetics can further include combinations of the above from either amides or esters.

The terms “anti-inflammatory” and “anti-inflammatory agent” are also used interchangeably herein, and mean and include a “pharmacological agent” and/or “active agent formulation”, which, when a therapeutically effective amount is administered to a subject, prevents or treats bodily tissue inflammation i.e. the protective tissue response to injury or destruction of tissues, which serves to destroy, dilute, or wall off both the injurious agent and the injured tissues.

Anti-inflammatory agents thus include, without limitation, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, decanoate, deflazacort, delatestryl, depo-testosterone, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenolone, methenolone acetate, methylprednisolone suleptanate, momiflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, triflumidate, zidometacin, and zomepirac sodium.

The term “pharmacological composition”, as used herein, means and includes a composition comprising a “pharmacological agent” and/or a “biologically active agent” and/or any additional agent or component identified herein.

The term “ECM composition”, as used herein, means and includes a composition comprising at least one ECM.

The term “therapeutically effective”, as used herein, means that the amount of the “pharmacological composition” and/or “pharmacological agent” and/or “biologically active agent” administered is of sufficient quantity to ameliorate one or more causes, symptoms, or sequelae of a disease or disorder. Such amelioration only requires a reduction or alteration, not necessarily elimination, of the cause, symptom, or sequelae of a disease or disorder.

The terms “prevent” and “preventing” are used interchangeably herein, and mean and include reducing the frequency or severity of a disease or condition. The term does not require an absolute preclusion of the disease or condition. Rather, this term includes decreasing the chance for disease occurrence.

The terms “treat” and “treatment” are used interchangeably herein, and mean and include medical management of a patient with the intent to cure, ameliorate, stabilize, or prevent a disease, pathological condition, or disorder. The terms include “active treatment”, i.e. treatment directed specifically toward the improvement of a disease, pathological condition, or disorder, and “causal treatment”, i.e. treatment directed toward removal of the cause of the associated disease, pathological condition, or disorder.

The terms “treat” and “treatment” further include “palliative treatment”, i.e. treatment designed for the relief of symptoms rather than the curing of the disease, pathological condition, or disorder, “preventative treatment”, i.e. treatment directed to minimizing or partially or completely inhibiting the development of the associated disease, pathological condition, or disorder, and “supportive treatment”, i.e. treatment employed to supplement another specific therapy directed toward the improvement of the associated disease, pathological condition, or disorder.

The terms “optional” and “optionally” mean that the subsequently described event, circumstance, or material may or may not occur or be present, and that the description includes instances where the event, circumstance, or material occurs or is present and instances where it does not occur or is not present.

The terms “patient” and “subject” are used interchangeably herein, and mean and include warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The term “comprise” and variations of the term, such as “comprising” and “comprises,” means “including, but not limited to” and is not intended to exclude, for example, other additives, components, integers or steps.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the pendency of this application and all equivalents of those claims as issued.

In overview, the present disclosure is directed to non-antigenic, resilient, bioremodelable, biocompatible mesh fiber members that can be engineered into a variety of shapes and used to repair, augment, or replace mammalian tissues and organs.

As indicated above, in some embodiments, the mesh fiber members comprise at least one biocompatible fiber member.

In some embodiments, the fiber member comprises a biocompatible fiber or strand.

In some embodiments, the strand comprises a bioremodelable strand.

In some embodiments, the strand comprises a biodegradable strand.

In some embodiments of the invention, the strand tensile strength is preferably in the range of approximately 200-1000 KPa.

In some embodiments of the invention, the Young's modulus of the strand is preferably in the range of approximately 30 to 400 KPa.

In some embodiments of the invention, the strand comprises at least one luminal cavity and/or recess.

In some embodiments, the strand comprises a porous member.

In some embodiments, the strand comprises a plurality of pores having a diameter in the range of approximately 0.1-100 μm.

In some embodiments, the strand comprises a ribbon.

According to the invention, the length of the strands can vary based on the fiber construct and/or mesh fiber member configuration.

In some embodiments, the mesh fiber members comprise a plurality of strands that define a fiber construct.

In some embodiments, the fiber constructs comprise a combination of biocompatible and/or biodegradable and/or bioremodelable strands.

According to the invention, the strands can be oriented in various configurations to form a fiber construct.

Thus, in some embodiments, the fiber constructs comprise an intertwined fiber or strand configuration.

In some embodiments, the fiber constructs comprise a braided strand configuration.

In some embodiments, the fiber constructs comprise a plurality of woven strands.

In some embodiments, the fiber constructs comprise a plurality of nonwoven strands.

In some embodiments, the fiber constructs comprise one of the aforementioned configurations having at least one strand and/or additional fiber construct wound about the outer surface of the fiber constructs.

According to the invention, the length of the fiber constructs can similarly vary based on the mesh fiber member configuration.

In some embodiments, the fiber constructs similarly have a tensile strength that is preferably in the range of approximately 200-1000 KPa.

In some embodiments, the Young's modulus of the fiber constructs is preferably in the range of approximately 30-400 KPa.

As indicated above, in some embodiments, the mesh fiber members of the invention comprise at least one fiber.

In some embodiments, the mesh fiber members comprise at least one fiber construct.

In some embodiments, the mesh fiber members comprise a plurality of strands and/or fiber constructs.

According to the invention, the strands and fiber constructs can be oriented in various configurations to form a mesh fiber member having a predetermined mesh pattern.

Thus, in some embodiments, the mesh pattern comprises an intertwined strand and/or fiber construct configuration.

In some embodiments, the mesh pattern comprises an intertwined directionally aligned strand and/or fiber construct configuration.

In some embodiments, the mesh pattern comprises an intertwined substantially perpendicular alignment of strands and/or fiber constructs.

In some embodiments, the mesh pattern comprises intertwined strands and/or fiber constructs that are oriented at an angle in the range of 0-90° relative to the plane defined by a linear axis of the mesh fiber member.

In some embodiments, the mesh pattern comprises a random orientation of strands and/or fiber constructs.

In some embodiments, the mesh pattern comprises intertwined strands and/or fiber constructs that are oriented at an angle in the range of 0-90° relative to the plane defined by a linear axis of the mesh fiber member.

As indicated above, the mesh fiber members can comprise virtually any desired height, width and/or length to accommodate various applications.

In a preferred embodiment of the invention, the mesh fiber members have a strand and/or fiber construct density “σ” in the range of approximately 10-90%.

In some embodiments, the mesh fiber members comprise an expansion ratio of approximately 1:2:1, more preferably, at least 2:1, even more preferably 3:1 when hydrated.

As also indicated above, in some embodiments of the invention, the mesh fiber members further comprise at least one secured border bound to at least one strand and/or fiber construct.

In some embodiments, the secured border comprises one of the aforementioned ECM or polymeric materials that is preferably treated by, without limitation, heat sealing, crosslinking (chemical, temperature, and/or photo-driven), suturing, biocompatible polymer glue, fibrin glue, and platelet-fibrin glue.

According to the invention, suitable crosslinking agents comprise, without limitation, gluteraldehyde, formaldehyde, phosphate buffered formalin, methanol, epoxides, genipin and/or derivatives thereof, carbodiimide compounds, polyepoxide compounds.

According to the invention, suitable agents for ECM crosslinking comprise, without limitation, transglutaminase, lysyl oxidase and riboflavin.

According to the invention, suitable photoinitiators for polymeric UV crosslinking comprise, without limitation, 2-hydroxy-1-[4-hydroxyethoxy)phenyl]-2-methyl-1-propanone (D 2959, Ciba Geigy), titanocenes, fluorinated diaryltitanocenes, iron arene complexes, manganese decacarbonyl, methylcyclopentadienyl manganese tricarbonyl and any organometallatic photoinitiator that produces free radicals or cations.

As further indicated above, in some embodiments of the invention, the strands and/or fiber constructs and, hence, mesh fiber constructs formed therefrom, comprise an ECM composition.

In a preferred embodiment of the invention, the ECM composition includes at least one extracellular matrix (hereinafter “ECM material”) derived from a mammalian tissue source. According to the invention, the ECM material can be derived from various mammalian tissue sources and methods for preparing same, such as disclosed in U.S. Pat. Nos. 7,550,004, 7,244,444, 6,379,710, 6,358,284, 6,206,931, 5,733,337 and 4,902,508 and U.S. application Ser. No. 12/707,427; which are incorporated by reference herein in their entirety. The mammalian tissue sources include, without limitation, the small intestine, large intestine, stomach, lung, liver, kidney, pancreas, placenta, heart, bladder, prostate, tissue surrounding growing enamel, tissue surrounding growing bone, and any fetal tissue from any mammalian organ.

The mammalian tissue can thus comprise, without limitation, small intestine submucosa (SIS), urinary bladder submucosa (UBS), stomach submucosa (SS), central nervous system tissue, epithelium of mesodermal origin, i.e. mesothelial tissue, dermal extracellular matrix, subcutaneous extracellular matrix, gastrointestinal extracellular matrix, i.e. large and small intestines, tissue surrounding growing bone, placental extracellular matrix, ornamentum extracellular matrix, cardiac extracellular matrix, e.g., pericardium and/or myocardium, kidney extracellular matrix, pancreas extracellular matrix, lung extracellular matrix, and combinations thereof. The ECM can also comprise collagen from mammalian sources.

In some embodiments, the mammalian tissue source comprises mesothelial tissue.

In a preferred embodiment, the mammalian tissue source comprises an adolescent mammalian tissue source, e.g. tissue derived from a porcine mammal less than 3 years of age.

The ECM can also be derived from the same or different mammalian tissue sources, as disclosed in Co-Pending application Ser. Nos. 13/033,053 and 13/033,102; which are incorporated by reference herein.

According to the invention, the ECM material can be used in whole or in part, so that, for example, an ECM material can contain just the basement membrane (or transitional epithelial layer) with the subadjacent tunica propria, the tunica submucosa, tunica muscularis, and tunica serosa. The ECM material component of the composition can contain any or all of these layers, and thus could conceivably contain only the basement membrane portion, excluding the submucosa. However, generally, and especially since the submucosa is thought to contain and support the active growth factors and other proteins necessary for in vivo tissue regeneration, the ECM or matrix composition from any given source will contain the active extracellular matrix portions that support cell development and differentiation and tissue regeneration.

According to the invention, the ECM material can comprise mixed solid particulates. The ECM material can also be formed into a particulate and fluidized, as described in U.S. Pat. Nos. 5,275,826, 6,579,538 and 6,933,326, to form a mixed emulsion, mixed gel or mixed paste.

According to the invention, the ECM can also be sterilized via applicant's proprietary novasterilis process disclosed in Co-Pending U.S. application Ser. No. 13/480,205; which is expressly incorporated by reference herein in its entirety.

In some embodiments, the ECM material is blended with an alginate to form an expandable composition having an expansion ratio of at least 5:1.

As indicated above, in some embodiments of the invention, the ECM compositions and/or materials and, hence, strands and fiber constructs formed therefrom, include at least one additional biologically active agent or composition, i.e. an agent that induces or modulates a physiological or biological process, or cellular activity, e.g., induces proliferation, and/or growth and/or regeneration of tissue.

Suitable biologically active agents include any of the aforementioned biologically active agents, including, without limitation, the aforementioned cells, proteins and growth factors.

In some embodiments, the ECM compositions and/or materials and, hence, mesh fiber members formed therefrom, include at least one pharmacological agent or composition (or drug), i.e. an agent or composition that is capable of producing a desired biological effect in vivo, e.g., stimulation or suppression of apoptosis, stimulation or suppression of an immune response, etc.

Suitable pharmacological agents and compositions include any of the aforementioned agents, including, without limitation, antibiotics, anti-viral agents, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

In some embodiments of the invention, the pharmacological agent comprises a statin, i.e. a HMG-CoA reductase inhibitor. According to the invention, suitable statins include, without limitation, atorvastatin (Lipitor®), cerivastatin, fluvastatin (Lescol®), lovastatin (Mevacor®, Altocor®, Altoprev®), mevastatin, pitavastatin (Livalo®, Pitava®), pravastatin (Pravachol®, Selektine®, Lipostat®), rosuvastatin (Crestor®), and simvastatin (Zocor®, Lipex®). Several actives comprising a combination of a statin and another agent, such as ezetimbe/simvastatin (Vytorin®), are also suitable.

Applicant has found that the noted statins exhibit numerous beneficial properties that provide several beneficial biochemical actions or activities. In particular, Applicant has found that when a statin is added to ECM (wherein a statin augmented ECM composition is formed) and the statin augmented ECM composition is administered to damaged tissue, the statin interacts with the cells recruited by the ECM, wherein the statin augmented ECM composition modulates inflammation of the damaged tissue by modulating several significant inflammatory processes, including restricting expression of monocyte chemoattractant protein-1 (MCP-1) and chemokine (C—C) motif ligand 2 (CCR2).

The properties and beneficial actions are discussed in detail in Applicant's Co-Pending application Ser. No. 13/328,287, filed on Dec. 16, 2011, Ser. No. 13/373,569, filed on Sep. 24, 2012 and Ser. No. 13/782,024, filed on Mar. 1, 2013; which are incorporated by reference herein in their entirety.

Additional suitable pharmacological agents and compositions that can be delivered within the scope of the invention are disclosed in Pat. Pub. Nos. 20070014874, 20070014873, 20070014872, 20070014871, 20070014870, 20070014869, and 20070014868; which are expressly incorporated by reference herein in its entirety.

According to the invention, the biologically active and pharmacological agents referenced above can comprise various forms. In some embodiments of the invention, the biologically active and pharmacological agents, e.g. simvastatin, comprise microcapsules that provide delayed delivery of the agent contained therein.

In some embodiments of the invention, the biologically active agent comprises a protein selected from the group comprising, without limitation, collagen (types I-V), proteoglycans, glycosaminoglycans (GAGs), glycoproteins, cytokines, cell-surface associated proteins, and cell adhesion molecules (CAMs).

In some embodiments, the biologically active agent provides a structural support scaffold. Suitable bioactive agents include, without limitation, elastin and ECM having additional GAG content, such as additional hyaluronic acid and/or chondroitin sulfate.

In some embodiments of the invention, the strands and fiber constructs and, hence, mesh fiber members formed therefrom comprise a polymeric composition comprising at least one biocompatible polymeric material.

According to the invention, the polymeric material can comprise, without limitation, polyglycolide (PGA), polylactide (PLA), polyepsilon-caprolactone (PCL), poly dioxanone (a polyether-ester), poly lactide-co-glycolide, polyamide esters, polyalkalene esters, polyvinyl esters, polyvinyl alcohol, and polyanhydrides. Natural polymeric materials, include, without limitation, polysaccharides (e.g. starch and cellulose), proteins (e.g., gelatin, casein, silk, wool, etc.), and polyesters (e.g., polyhydroxyalkanoates).

The polymeric material can also comprise a hydrogel, including, without limitation, polyurethane, poly(ethylene glycol), poly(propylene glycol), poly(vinylpyrrolidone), xanthan, methyl cellulose, carboxymethyl cellulose, alginate, hyaluronan, poly(acrylic acid), polyvinyl alcohol, acrylic acid, hydroxypropyl methyl cellulose, methacrylic acid, αβ-glycerophosphate, κ-carrageenan, 2-acrylamido-2-methylpropanesulfonic acid, and β-hairpin peptide.

In some embodiments, the hydrogel is crosslinked via chemically and/or photocuring, e.g. ultraviolet light.

In some embodiments, the polymeric material is plasma treated to accommodate hygroscopic agents.

In some embodiments, the polymeric composition includes at least one of the aforementioned biologically active or pharmacological agents.

In some embodiments of the invention, the strands and fiber constructs and, hence, mesh fiber members formed therefrom comprise an ECM-mimicking biomaterial.

In some embodiments, the ECM-mimicking biomaterial comprises poly(glycerol sebacate) (PGS).

Applicant has found that PGS exhibits numerous beneficial properties that provide several beneficial biochemical actions or activities. The properties and beneficial actions resulting therefrom are discussed in detail below.

### PGS Physical Properties

PGS is a condensate of the non-immunogenic compositions glycerol (a simple sugar alcohol) and sebacic acid (a naturally occurring dicarboxylic acid), wherein, glycerol and sebacic acid are readily metabolized when proximate mammalian tissue. The non-immunogenic properties substantially limit the acute inflammatory responses typically associated with other “biocompatible” polymers, such as ePTFE (polytetrafluoroethylene), that are detrimental to bioremodeling and tissue regeneration.

The mechanical properties of PGS are substantially similar to that of biological tissue, wherein, the value of the Young's modulus of PGS is between that of a ligament (in KPa range) and tendon (in GPa range). The strain to failure of PGS is also similar to that of arteries and veins (i.e. over 260% elongation).

The tensile strength of the PGS is at least 0.28±0.004 MPa. The Young's modulus and elongation are at least 0.122±0.0003 and at least 237.8±0.64%, respectively. For applications requiring stronger mechanical properties and a slower biodegradation rate, PGS can be blended with PCL, i.e. a biodegradable elastomer.

### ECM Mimicking Properties/Actions

It has been found that PGS induces tissue remodeling and regeneration when administered proximate to damaged tissue, thus, mimicking the seminal regenerative properties of ECM and, hence, an ECM composition formed therefrom. The mechanism underlying this behavior is deemed to be based on the mechanical and biodegradation kinetics of the PGS. See Sant, et al., Effect of Biodegradation and de novo Matrix Synthesis on the Mechanical Properties of VIC-seeded PGS-PCL scaffolds, Acta. Biomater., vol. 9(4), pp. 5963-73 (2013).

In some embodiments of the invention, the strands and fiber constructs and, hence, mesh fiber members formed therefrom comprise an ECM-mimicking composition comprising PGS and PCL. According to the invention, the addition of PCL to the ECM-mimicking composition enhances the structural integrity and modulates the degradation of the composition.

In some embodiments of the invention, the strands and fiber constructs and, hence, mesh fiber members formed therefrom comprise an ECM-PGS composition, e.g. 50% ECM/50% PGS.

In some embodiments, the ECM-PGS composition further comprises PCL.

In some embodiments, the ECM-mimicking biomaterial, ECM mimicking composition and/or ECM-PGS composition include at least one of the aforementioned biologically active or pharmacological agents.

In some embodiments, the fiber constructs comprise a blended plurality of ECM, polymeric, ECM-mimicking biomaterial, and/or ECM-PGS composition strands.

In some embodiments, the mesh fiber members comprise a blend of strands and/or fiber constructs having a plurality of ECM, polymeric, ECM-mimicking biomaterial, and/or ECM-PGS composition strands and/or fiber constructs.

In some embodiments, the plurality of blended ECM, polymeric, ECM-mimicking biomaterial, and/or ECM-PGS composition strands and/or fiber constructs includes one of the aforementioned biologically active or pharmacological agents.

In some embodiments, the strands and/or fiber constructs and, hence, mesh fiber members formed therefrom provide a single-stage agent delivery profile, i.e. comprise a single-stage delivery vehicle, wherein a modulated dosage of an aforementioned biologically active and/or pharmacological agent is provided.

According to the invention, the term “modulated dosage” as used herein, and variants of this language generally refer to the modulation (e.g., alteration, delay, retardation, reduction, etc.) of a process involving different eluting or dispersal rates of an agent within biological tissue.

In some embodiments, the single-stage delivery vehicle comprises encapsulated particulates of a biologically active and/or pharmacological agent.

In some embodiments, the encapsulation composition comprises an ECM composition.

In some embodiments, the encapsulation composition comprises a biodegradable polymeric composition comprising a polymeric material selected from the group comprising, without limitation, polyglycolide (PGA), polylactide (PLA), polyepsilon-caprolactone, poly-dioxanone, poly lactide-co-glycolide polysaccharides (e.g. starch and cellulose), proteins (e.g., gelatin, casein, silk, wool, etc.), one of the aforementioned hydrogels, and combinations thereof.

In some embodiments of the invention, the encapsulation composition comprises an ECM-mimicking biomaterial.

In some embodiments of the invention, the encapsulation composition comprises an ECM-mimicking composition.

In some embodiments of the invention, the encapsulation composition comprises an ECM-PGS composition.

In some embodiments, the encapsulation composition comprises an osmotic fluctuation inducing composition. According to the invention, suitable osmotic fluctuation inducing compositions include, without limitation, polyethylene glycol, alginate and dextran.

According to the invention, the term “osmotic fluctuation” as used herein, and variants of this language generally refer to the modulation of the osmotic pressure gradient across a defined barrier.

For example, as is well known in the art, alginate is capable of absorbing 200-300 times its weight in water, which substantially increases the osmotic pressure gradient of the alginate. The increased osmotic pressure gradient of the alginate results in a rapid dispersal of an agent therefrom.

In some embodiments of the invention, the strands and/or fiber constructs and, hence, mesh fiber members formed therefrom provide a multi-stage agent delivery profile, i.e. comprise a multi-stage agent delivery vehicle, wherein a plurality of the aforementioned biologically active and/or pharmacological agents are administered via a modulated dosage. By way of example, in some embodiments, the multi-stage delivery vehicle comprises encapsulated particulates comprising an antibiotic composition encapsulated in an alginate composition having a statin incorporated therein, which provides a tiered modulated agent delivery.

In some embodiments, the multi-stage agent delivery vehicle comprises a combination of different biologically active and/or pharmacological agents. By way of example, in some embodiments, the multi-stage delivery vehicle comprises encapsulated particulates comprising an encapsulated growth factor concomitantly administered with an encapsulated anti-inflammatory.

In some embodiments, the multi-stage delivery vehicle comprises a plurality of different biologically active and/or pharmacological agents encapsulated in different encapsulation compositions. By way of example, in some embodiments, the multi-stage delivery vehicle comprises encapsulated particulates comprising a growth factor encapsulated in alginate composition and a pharmacological agent encapsulated in a polyglycolide composition.

In some embodiments, the mesh fiber members comprise at least one coating. In some embodiments, the coating includes at least one of the aforementioned biologically active and/or pharmacological agents.

In some embodiments, the coating is applied to the individual strands and/or fiber constructs that are employed to construct the mesh fiber member or directly applied to mesh fiber member.

In some embodiments, the individual strands, fiber constructs and/or mesh fiber member comprise the same coating.

In some embodiments, the individual strands, fiber constructs and/or mesh fiber member comprise a combination of coatings having varying biologically active and/or pharmacological agents and/or properties, e.g. a first coating comprising a growth factor and a second coating comprising pharmacological agent.

In some embodiments, the strands comprise at least one intraluminal coating that includes at least one of the aforementioned biologically active or pharmacological agents.

In some embodiments, the coating comprises modulated degradation kinetics, wherein the gradual degradation of the coating provides a controlled release of biologically active and/or pharmacological agents.

In some embodiments, the coating comprises an ECM composition.

According to the invention, suitable ECM compositions comprising ECM, and ECM and biologically active and/or pharmacological agents are disclosed in U.S. Pat. Nos. 8,568,761, 8,753,885, 8,795,728, 8,734,841, 8,642,084, 8,771,737, 8,734,842, 8,784,891, 8,753,886, 8,785,197, 8,785,198, 8,735,155 and U.S. patent application Ser. No. 13/732,943, filed on Jan. 2, 2013, Ser. No. 11/448,351, filed on Jun. 6, 2006, Ser. No. 14/269,324, filed on May 5, 2014, Ser. No. 13/732,558, filed on Jan. 2, 2013, Ser. No. 13/732,731, filed on Jan. 2, 2013, Ser. No. 13/875,017, filed on May 1, 2013, Ser. No. 13/875,043, filed on May 1, 2013, Ser. No. 13/875,058, filed on May 1, 2013, Ser. No. 14/452,707, filed on Aug. 6, 2014, Ser. No. 14/192,973, filed on Jan. 28, 2014, Ser. No. 14/192,992, filed on Feb. 28, 2014, Ser. No. 14/193,008, filed on Feb. 28, 2014, Ser. No. 14/193,030, filed on Feb. 28, 2014, Ser. No. 14/193,053, filed on Feb. 28, 2014, Ser. No. 14/269,414, filed on Mar. 3, 2013, Ser. No. 14/269,487, filed on Mar. 3, 2013, Ser. No. 14/269,874, filed on Mar. 3, 2013, Ser. No. 14/337,460, filed on Mar. 3, 2013; which are incorporated by reference herein in their entirety.

In some embodiments, the ECM coating is configured to provide at least one biologically active and/or pharmacological agent delivery profile.

In some embodiments, the ECM coating is configured to provide a delivery gradient of various biologically active and/or pharmacological agent delivery profiles. By way of example, in some embodiments, biologically active and/or pharmacological agents are disposed throughout various depths or thickness ranges of the ECM coating.

In some embodiments, the plurality of ECM coatings is configured to provide a plurality of biologically active and/or pharmacological agent delivery profiles. By way of example, in some embodiments, the mesh fiber member members comprise a coating comprising a growth factor augmented ECM composition, and the mesh constructs formed therefrom include an ECM composition comprising a pharmacological agent, such as an anti-inflammatory or antiviral.

In some embodiments, the strands, fiber constructs and/or mesh fiber members comprise an ECM coating comprising anti-inflammatory growth factors interleukin-10 (IL-10) and transforming growth factor beta (TGF-β) either alone, or in combination, to suppress the inflammatory reaction leading to a chronic immune response. During the chronic immune response IL-10 and TGF-β induce the expression of tissue inhibitor of metalloproteinase (TIMP), which inhibits matrix metalloproteinases (MMPs) that are responsible for ECM degradation during the inflammatory response. Additionally, IL-10 and TGF-β promote the recruitment of fibroblasts, which are the seminal cells responsible for ECM deposition and bioremodeling. As a result, IL-10, TGF-β, and the TIMPs concomitantly promote ECM deposition and preservation, which thus augments “modulated healing.”

In some embodiments, the strands, fiber constructs and/or mesh fiber members comprise an ECM coating comprising a pharmacological agent, such as an anti-inflammatory or antiviral, which provide a reinforcing anti-inflammatory effect either through direct reinforcement, i.e. targeting the same inflammatory signaling pathway, or indirect reinforcement, i.e. targeting an alternate inflammatory signaling pathway. An example of direct reinforcement includes, without limitation, a combination of IL-10, TGF-13 and a glucocorticoid, all of which inhibit the expression of seminal inflammatory cytokine interleukin-1 (IL-1). An example of indirect reinforcement includes, without limitation, a combination of IL-10, TGF-β and an NSAID, (Non-steroidal anti-inflammatory drug) where IL-10 and TGF-β inhibit IL-1, and the NSAIDs inhibit the activity of both cyclooxygenase-1 (COX-1) and cyclooxygenase-2 (COX-2), and thereby, the synthesis of prostaglandins and thromboxanes.

In some embodiments of the invention, the coating comprises a polymeric composition comprising at least one of the aforementioned polymeric materials.

In some embodiments, the coating comprises an ECM-mimicking biomaterial.

In some embodiments of the invention, the coating comprises an ECM-mimicking composition.

In some embodiments of the invention, the coating comprises an ECM-PGS composition.

In some embodiments, the polymeric, ECM-mimicking biomaterial, and/or ECM-PGS composition coating(s) are similarly configured to provide at least one biologically active and/or pharmacological agent delivery profile. Again, by way of example, in some embodiments, the mesh fiber members comprise a polymeric coating comprising a growth factor augmented polymeric composition, and the mesh fiber constructs therefrom comprise a polymeric composition comprising a pharmacological agent, such as an anti-inflammatory or antiviral.

In some embodiments, the delivery profile comprises modulated degradation, i.e. a drug infused hydrogel coating configured to provide a precise dosage of biologically active and/or pharmacological agents based on the biological half-life of the material.

According to the invention, the coatings can additionally comprise a hydrogel, including, without limitation, polyurethane, poly(ethylene glycol), poly(propylene glycol), poly(vinylpyrrolidone), xanthan, methyl cellulose, carboxymethyl cellulose, alginate, hyaluronan, poly(acrylic acid), polyvinyl alcohol, acrylic acid, hydroxypropyl methyl cellulose, methacrylic acid, αβ-glycerophosphate, κ-carrageenan, 2-acrylamido-2-methylpropanesulfonic acid, and β-hairpin peptide. In some embodiments, the hydrogels are similarly configured to provide at least one biologically active and/or pharmacological agent delivery profile.

In some embodiments, the coating comprises a blend of the aforementioned ECM and polymeric materials and/or ECM-mimicking biomaterials and/or ECM-PGS compositions.

In some embodiments, the ECM, polymeric, ECM-mimicking biomaterial, and/or ECM-PGS composition blended coating(s) are similarly configured to provide at least one biologically active and/or pharmacological agent delivery profile. By way of example, in some embodiments, the mesh fiber members comprise a first coating comprising a growth factor augmented polymer/ECM blended composition, and the mesh constructs therefrom comprise a ECM-PGS composition comprising a pharmacological agent, such as an anti-inflammatory or antiviral.

In some embodiments, the delivery profile similarly comprises modulated degradation, e.g. a drug infused ECM/hydrogel blended coating configured to provide a first dosage of biologically active and/or pharmacological agents based on the biological half-life of the material.

In some embodiments, the coating comprises a thickness in the range of 5-100 μm, which can vary based on the orientation and the size of the mesh fiber members and/or fiber constructs. In some embodiments, the coating thickness is in the range of 10-20 μm. If multiple coatings are employed, the total coating thickness is preferably in the range of 5-200 μm, more preferably, in the range of 50-80 μm.

In some embodiments, the coatings provide a multi-stage strength profile.

In some embodiments, the multi-stage strength profile comprises a biodegradable coating providing a tensile strength at least 10% greater than the mesh fiber members and fiber constructs.

In a preferred embodiment, the multi-stage strength profile provides a greater tensile strength during the initial stage of the healing process, while progressively yielding a substantially more malleable structure during bioremodeling.

According to the invention, upon deployment of a strand or fiber construct comprising ECM, an ECM-PGS composition and polymeric composition comprising exogenously added biologically active and/or pharmacological agents, and, hence, a mesh fiber member formed therefrom to damaged biological tissue, “modulated healing” is effectuated.

The term “modulated healing”, as used herein, and variants of this language generally refer to the modulation (e.g., alteration, delay, retardation, reduction, etc.) of a process involving different cascades or sequences of naturally occurring tissue repair in response to localized tissue damage or injury, substantially reducing their inflammatory effect. Modulated healing, as used herein, includes many different biologic processes, including epithelial growth, fibrin deposition, platelet activation and attachment, inhibition, proliferation and/or differentiation, connective fibrous tissue production and function, angiogenesis, and several stages of acute and/or chronic inflammation, and their interplay with each other.

For example, in some embodiments, the strands and/or fiber constructs, and, hence, mesh fiber members formed therefrom are specifically formulated (or designed) to alter, delay, retard, reduce, and/or detain one or more of the phases associated with healing of damaged tissue, including, but not limited to, the inflammatory phase (e.g., platelet or fibrin deposition), and the proliferative phase when in contact with biological tissue.

In some embodiments of the invention, “modulated healing” means and includes the ability of a strand and/or fiber construct, and, hence, mesh fiber member formed therefrom to restrict the expression of inflammatory components. By way of example, according to the invention, when a strand and/or fiber construct, and, hence, mesh fiber member formed therefrom comprises a statin augmented ECM composition, i.e. a composition comprising an ECM and an exogenously added statin, is disposed proximate damaged biological tissue, the strand and/or fiber construct and/or mesh fiber member restricts expression of monocyte chemoattractant protein-1 (MCP-1) and chemokine (C—C) motif ligand 2 (CCR2).

In some embodiments, “modulated healing” means and includes the ability of a strand and/or fiber construct, and, hence, mesh fiber member formed therefrom to alter a substantial inflammatory phase (e.g., platelet or fibrin deposition) at the beginning of the tissue healing process. As used herein, the phrase “alter a substantial inflammatory phase” refers to the ability of a mesh fiber member to substantially reduce the inflammatory response at an injury site when in contact with biological tissue.

In such an instance, a minor amount of inflammation may ensue in response to tissue injury, but this level of inflammation response, e.g., platelet and/or fibrin deposition, is substantially reduced when compared to inflammation that takes place in the absence of a mesh fiber member of the invention.

For example, several strands discussed herein have been shown experimentally to delay or alter the inflammatory response associated with damaged tissue, as well as excessive formation of connective fibrous tissue following tissue damage or injury. The mesh fiber members have also been shown experimentally to delay or reduce fibrin deposition and platelet attachment to a blood contact surface following tissue damage.

The term “modulated healing” also refers to the ability of a strand and/or fiber construct, and, hence, mesh fiber member formed therefrom to induce host tissue proliferation, bioremodeling, including neovascularization, e.g., vasculogenesis, angiogenesis, and intussusception, and regeneration of tissue structures with site-specific structural and functional properties.

Thus, in some embodiments, the term “modulated healing” means and includes the ability of a strand and/or fiber construct, and, hence, mesh fiber member formed therefrom to modulate inflammation and/or induce host tissue proliferation and remodeling. Again, by way of example, according to the invention, when a strand and/or fiber construct, and, hence, mesh fiber member formed therefrom comprises a statin augmented ECM composition, i.e. a composition comprising an ECM and an exogenously added statin, is disposed proximate damaged biological tissue, the stain interacts with cells recruited by the ECM, wherein the strand and/or fiber construct and/or mesh fiber member modulates inflammation by, among other actions, restricting expression of monocyte chemoattractant protein-1 (MCP-1) and chemokine (C—C) motif ligand 2 (CCR2) and induces tissue proliferation, bioremodeling and regeneration of tissue structures with site-specific structural and functional properties.

By way of a further example, according to the invention, when a strand and/or fiber construct, and, hence, mesh fiber member formed therefrom comprises a growth factor augmented ECM composition, i.e. a composition comprising an ECM and an exogenously added growth factor, e.g. TGF-β, is disposed proximate damaged biological tissue, the growth factor similarly interacts with the ECM and cells recruited by the ECM, wherein the strand and/or fiber construct and/or mesh fiber member modulates inflammation and induces tissue proliferation, bioremodeling and regeneration of tissue.

In some embodiments, when a mesh fiber member is in contact with biological tissue modulated healing is effectuated through the structural features of a mesh fiber member. The structural features provide the spatial temporal and mechanical cues to modulate cell polarity and alignment. The structural features further modulate cell proliferation, migration and differentiation thus modulating the healing process.

In some embodiments, the mesh fiber members comprise an anisotropic fiber structure providing spatial temporal and mechanical cues.

Accordingly, the mesh fiber members of the invention provide an excellent means for treating damaged or diseased biologically tissue, including closing and maintaining closure of openings in biological tissue, e.g., closure of openings in tissue after surgical intervention.

According to the invention, various conventional methods can be employed to form and/or extrude the aforementioned mesh fiber members of the invention, including, without limitation, break spinning, open-end spinning, melt spinning, dry spinning, wet spinning, coaxial electrospinning, needleless electrospinning, and Forcespinning®.

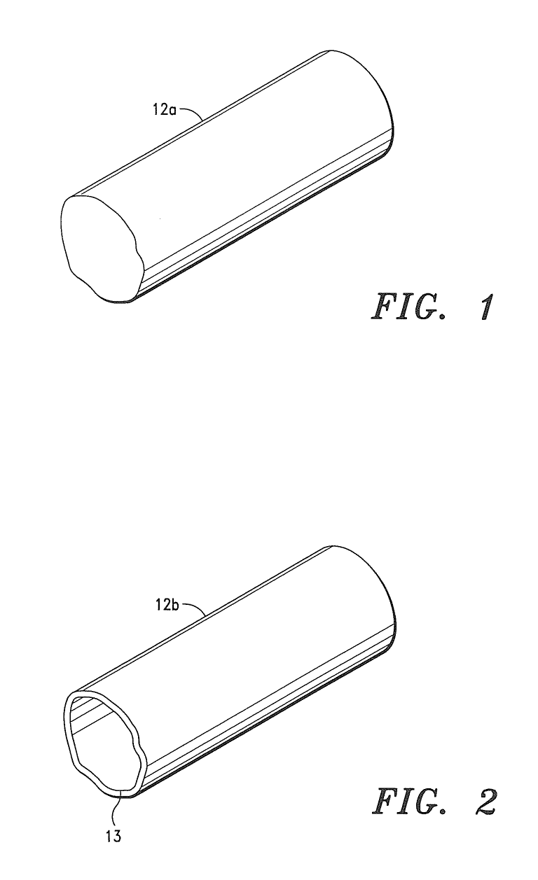

Referring now to FIG. 1, there is shown one embodiment of a biocompatible strand 12a of the invention. As indicated above, the strand 12a can comprise various dimensions, e.g., length, circumference, etc., to accommodate various fiber construct and mesh fiber member structures and applications.

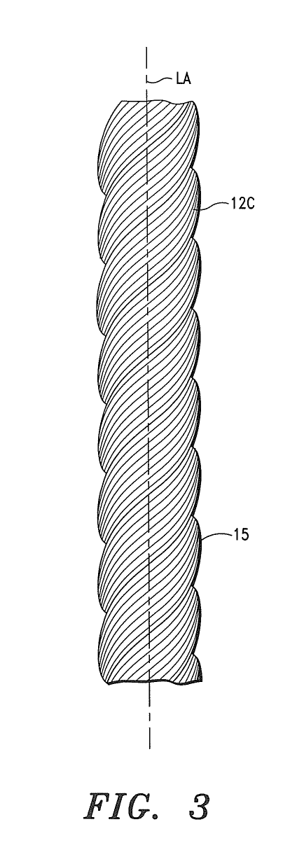

Referring now to FIG. 2, there is shown another embodiment of a biocompatible strand 12b of the invention. As illustrated in FIG. 2, the strand 12b includes a luminal cavity 13.

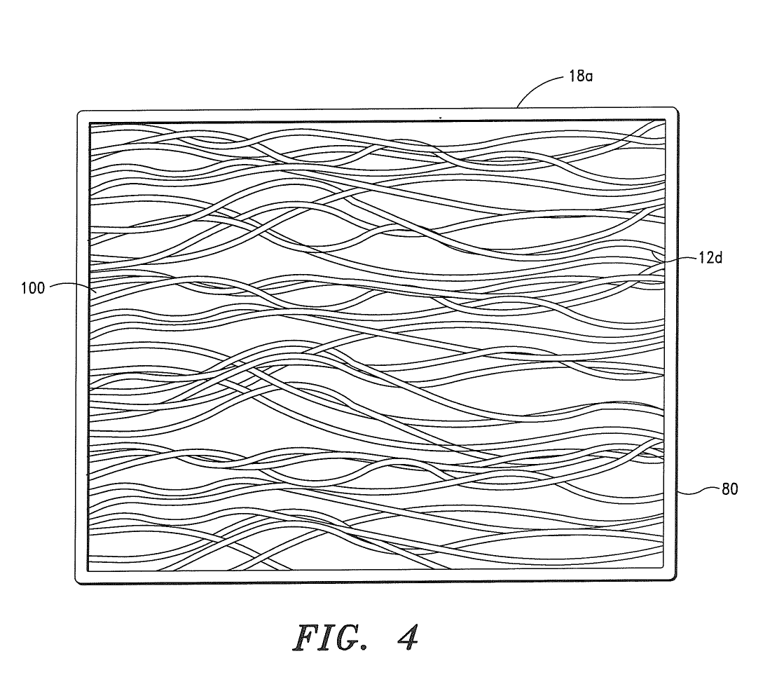

Referring now to FIG. 3, there is shown one embodiment of a fiber construct 15 of the invention. As illustrated in FIG. 3, the fiber construct 15 comprises a plurality of strands 12c, arranged in a substantially braided structure.

According to the invention, the fiber construct 15 can similarly comprise various dimensions to accommodate various mesh fiber member structures and application.

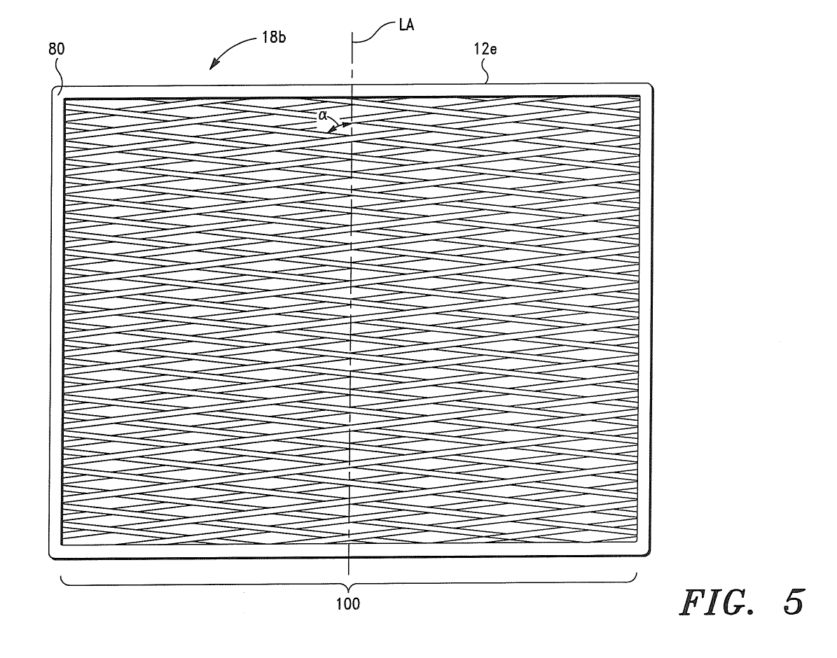

Referring now to FIG. 4, there is shown one embodiment of the mesh fiber member 18a of the invention. As illustrated in FIG. 4, the mesh fiber member 18a comprises a plurality of interwoven or intersecting strands 12d. As further illustrated in FIG. 4, the mesh fiber member 18a further comprises a constraining edge or border 80 that forms an internal fiber region 100.

According to the invention, the mesh fiber member 18a can also comprise a plurality of fiber constructs, such as construct 15 as shown in FIG. 3.

According to the invention, the mesh fiber member 18a can similarly comprise various dimensions to accommodate various structures and application.

Referring now to FIG. 5, there is shown another embodiment of a mesh fiber member 18b. As illustrated in FIG. 5, in this embodiment, the mesh fiber member 18b comprises a plurality of intertwined strands 12e.

In the illustrated embodiment, each strand 12e, is oriented at an angle (“a”) in the range of approximately 0-89° relative to a line corresponding to the plane defined by the linear axis (“LA”) of the member 18b.

Referring now to FIG. 6, there is shown another embodiment of a mesh fiber member 18c having a plurality of substantially perpendicular interwoven or intersecting strands 12f.

Referring now to FIG. 7, there is shown another embodiment of the mesh fiber member 18e having a plurality of nonwoven or intersecting strands 12a.

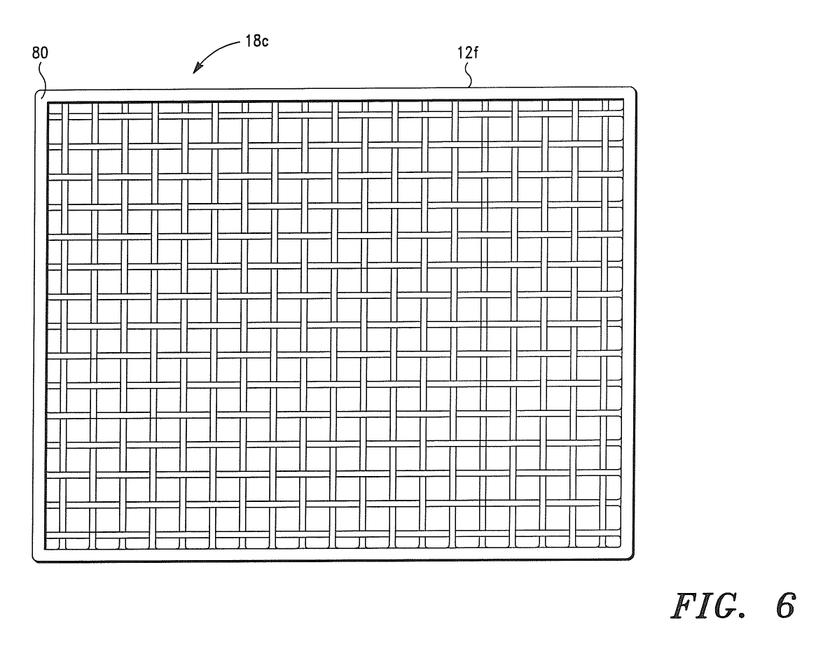

Referring now to FIG. 8, there is shown another embodiment of the mesh fiber member 18d having a plurality of intertwined, randomly oriented strands 12g.

It is understood that the mesh fiber member patterns shown in FIGS. 4-7 are merely examples of the various mesh patterns that can be employed within the scope of the invention. The mesh patterns shown in FIGS. 4-7 should thus not be construed as limiting the scope of the invention in any manner.

As will be readily appreciated by one having ordinary skill in the art, the mesh fiber members of the invention can be readily employed in various medical procedures, including, without limitation, treatment of coronary and peripheral vascular disease (PVD) in cardiovascular vessels, including, but not limited to, iliacs, superficial femoral artery, renal artery, tibial artery, popliteal artery, etc., deep vein thromboses (DVT), vascular bypasses, and coronary vascular repair.

The mesh fiber members can also be readily employed to construct a biomaterial pouch that is configured to encase an ECM or pharmacological composition, or medical instrument or device, such as a pacemaker, therein. Illustrative pouch configurations are disclosed U.S. Pat. No. 8,758,448 and Applicant's Co-pending U.S. application Ser. Nos. 13/573,566 and 13/896,424, which are incorporated by reference herein in their entirety.

### EXAMPLES

The following examples are provided to enable those skilled in the art to more clearly understand and practice the present invention. They should not be considered as limiting the scope of the invention, but merely as being illustrated as representative thereof.

### Example 1

An ECM patch (i.e. matrix) comprising small intestine submucosa (SIS) and 1 mg/ml of a statin, i.e. cerivastatin, was surgically applied to the myocardium of two canines. The ECM patches remained attached to the myocardium of the canines until they were sacrificed at 2 and 24 hours, respectively.

Cardiac tissue samples were collected immediately after the canines were sacrificed. The cardiac tissue samples were then subjected to mRNA extraction and quantification via established protocols.

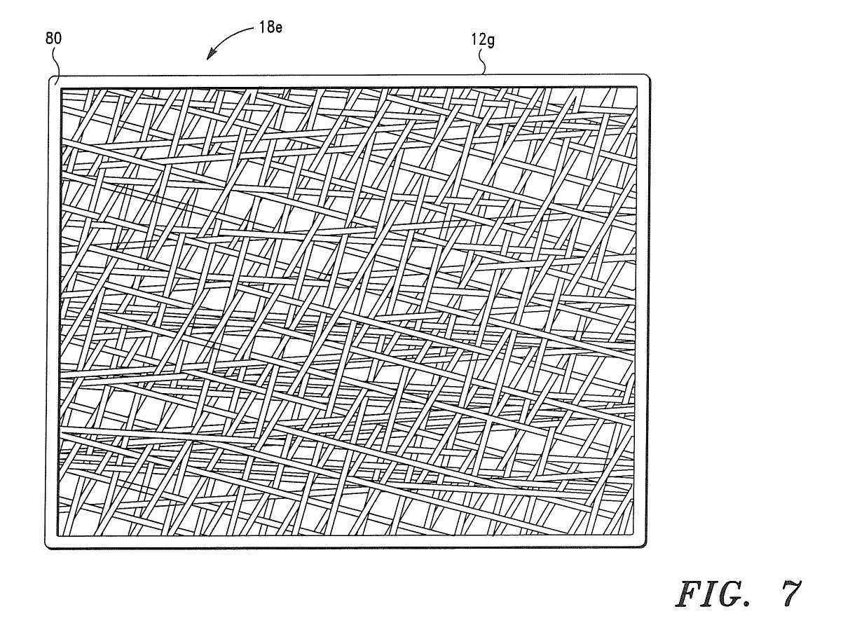

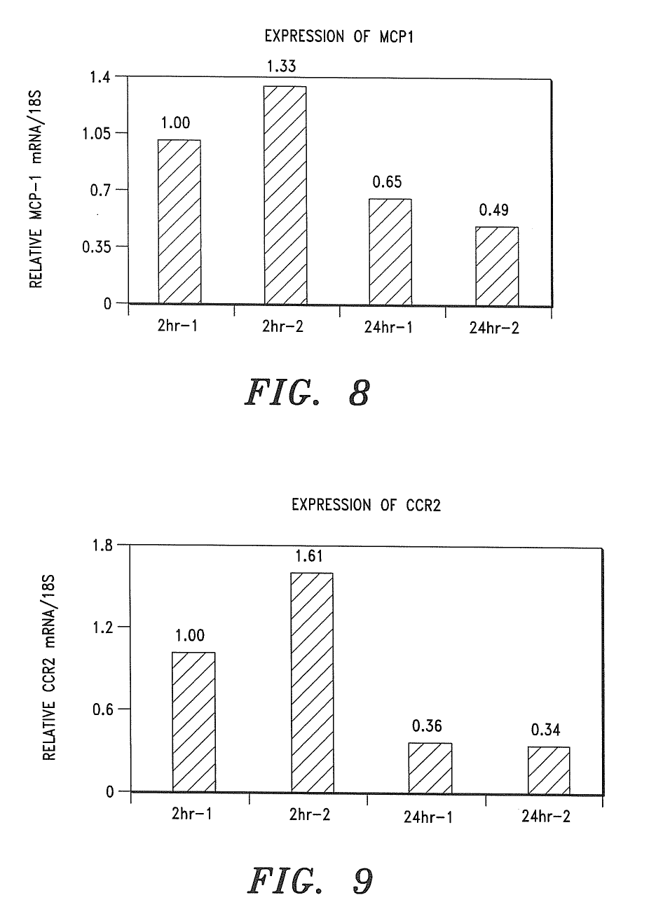

The measured mRNA levels from the cardiac tissue samples, which are shown in FIGS. 8-10, reflect substantially reduced MCP-1 and CCR2 expression at a 24 hour time point compared to the MCP-1 and CCR2 expression at a 2 hour time point. The mRNA levels thus reflect a consistent and highly effective anti-inflammatory effect over time in vivo, when a statin augmented ECM is administered to biological tissue.

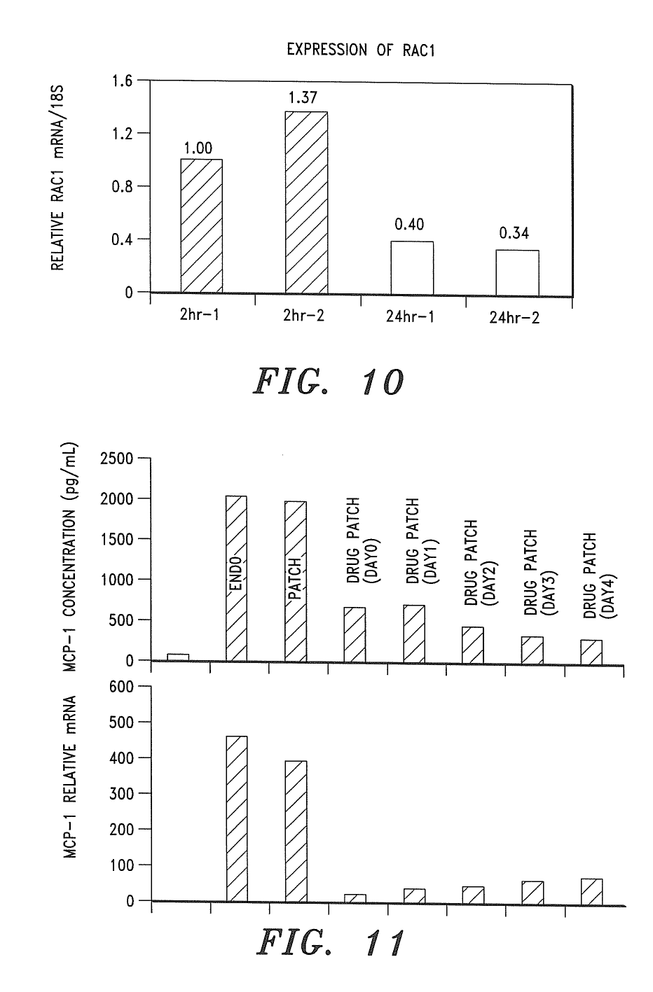

The canine model experiment was further reinforced by an additional in vitro study, wherein MCP-1 expression of THP-1 cells (a human monocytic cell line) in the presence of a statin augmented ECM was analyzed. As reflected in FIG. 11, the statin augmented ECM induced substantially lower MCP-1 expression when compared to a positive control.

The example thus confirms that when a statin augmented ECM and, hence, strand and/or fiber construct and, hence, mesh fiber member formed therefrom, is administered to damaged tissue, the strand and/or fiber construct and, hence, mesh fiber member formed therefrom modulates several significant inflammation processes, including inhibiting generation of MCP-1 and CCR2.

The example further established that when a statin augmented ECM and, hence, strand and/or fiber construct and, hence, mesh fiber member formed therefrom, is administered to damaged tissue, the strand and/or fiber construct and, hence, mesh fiber member formed therefrom induces tissue proliferation and remodeling.

One having ordinary skill in the art will thus readily appreciate that the mesh fiber members of the invention provide numerous advantages over conventional apparatus and structures for repairing and/or regenerating tissue. Among the advantages are the following:
The provision of mesh fiber members that can be readily and effectively employed to treat damaged or diseased biological tissue; particularly, cardiovascular tissue;
The provision of mesh fiber members that can be readily employed to close and maintain closure of openings in biological tissue;
The provision of mesh fiber members having a plurality of fibers configured to provide spatial and mechanical cues that modulate cell polarity, spatial temporal positioning, differentiation, proliferation and migration when in contact with biological tissue; particularly, damaged and/or diseased tissue cells;
The provision of mesh fiber members that induce host tissue proliferation, bioremodeling and regeneration of new tissue, and tissue structures with site-specific structural and functional properties; and
The provision of mesh fiber members that effectively administer at least one biologically active agent and/or pharmacological agent or composition to a subject's tissue to induce a desired biological and/or therapeutic effect.

Without departing from the spirit and scope of this invention, one of ordinary skill can make various changes and modifications to the invention to adapt it to various usages and conditions. As such, these changes and modifications are properly, equitably, and intended to be, within the full range of equivalence of any issued claims.

1. An implantable mesh construct for treating damaged biological tissue, comprising:
a mesh structure comprising a plurality of biodegradable fibers, each of said plurality of biodegradable fibers having an outer surface,
said plurality of biodegradable fibers comprising an extracellular matrix (ECM) composition comprising acellular ECM material from a mammalian tissue source, said mammalian tissue source comprising mesothelial tissue,
at least one of said plurality of biodegradable fibers comprising an outer coating comprising an ECM-mimicking biomaterial composition, said ECM-mimicking biomaterial composition coating being disposed on said outer surface of said fiber,
wherein said mesh structure induces modulated healing when implanted in host tissue of a subject's body, said modulated healing comprising modulation of inflammation of said host tissue, and induced tissue proliferation and bioremodeling of said host tissue.

2. The mesh construct of claim 1, wherein said ECM composition further comprises a statin selected from the group consisting of atorvastatin, cerivastatin, fluvastatin, lovastatin, mevastatin, pitavastatin, pravastatin, rosuvastatin and simvastatin.

3. The mesh construct of claim 1, wherein said ECM composition further comprises a first exogenously added biologically active agent.

4. The mesh construct of claim 3, wherein said first exogenously added biologically active agent comprises a growth factor selected from the group consisting of transforming growth factor alpha (TGF-α), transforming growth factor beta (TGF-β), fibroblast growth factor-2 (FGF-2), vascular epithelial growth factor (VEGF), and insulin-like growth factor (IGF).

5. The mesh construct of claim 3, wherein said first exogenously added biologically active agent comprises a cell selected from the group consisting of an embryonic stem cell, mesenchymal stem cell, hematopoietic stem cell, bone marrow stem cell, bone marrow-derived progenitor cell, myosatellite progenitor cell, totipotent stem cell, pluripotent stem cell, multipotent stem cells, oligopotent stem cell and unipotent stem cell.

6. The mesh construct of claim 3, wherein said first exogenously added biologically active agent comprises a protein selected from the group consisting of collagen, proteoglycans, glycosaminoglycans (GAGs), glycoproteins, cytokines, cell-surface associated proteins, and cell adhesion molecules (CAMs).

7. The mesh construct of claim 1, wherein said mammalian tissue source comprises an adolescent mammalian tissue source.

8-17. (canceled)

18. The mesh structure of claim 1, wherein said ECM-mimicking biomaterial composition comprises poly(glycerol sebacate) (PGS).

19. The mesh structure of claim 1, wherein said ECM-mimicking biomaterial composition comprises poly(glycerol sebacate) (PGS) and PCL.

20. (canceled)

21. The mesh structure of claim 1, wherein said ECM-mimicking biomaterial composition further comprises at least one second biologically active agent.

22. The mesh structure of claim 21, wherein said second biologically active agent comprises a growth factor selected from the group consisting of transforming growth factor alpha (TGF-α), transforming growth factor beta (TGF-β), fibroblast growth factor-2 (FGF-2), vascular epithelial growth factor (VEGF), and insulin-like growth factor (IGF).

23. The mesh structure of claim 1, wherein said ECM-mimicking biomaterial composition further comprises at least one pharmacological agent.

24. The mesh structure of claim 23, wherein said pharmacological agent comprises an agent selected from the group consisting of an anti-viral agent, analgesic, antibiotic, anti-inflammatory, anti-neoplastic agent, anti-spasmodic agent, enzyme, anticoagulant, antithrombic agent, and vasodilating agent.
